(19) United States
(12) Patent Application Publication
(10) Pub. No.: US 2019/0133977 A1
(43) Pub. Date: May 9, 2019

(54) DELAYED RELEASE ORAL TAMSULOSIN HYDROCHLORIDE

(71) Applicant: ASPEN PARK PHARMACEUTICALS, INC., Scarsdale, NY (US)

(72) Inventors: K. Gary Barnette, Wake Forest, NC (US); Ruth E. Stevens, Cincinnati, OH (US); Kenneth V. Phelps, Cincinnati, OH (US); Lynn Gold, Seattle, WA (US)

(73) Assignee: ASPEN PARK PHARMACEUTICALS, INC., Scarsdale, NY (US)

(21) Appl. No.: 16/099,731

(22) PCT Filed: May 4, 2017
(86) PCT No.: PCT/US2017/030989

§ 371 Date: Nov. 8, 2018

Related U.S. Application Data
(60) Provisional application No. 62/331,599, filed on May 4, 2016.

(51) Int. Cl. A61K 31/18; A61K 9/00; A61K 9/16

(52) U.S. Cl. CPC A61K 31/18; A61K 9/1652; A61K 9/0095

(57) ABSTRACT

The present invention relates in certain embodiments to a controlled release formulation, especially a sachet, comprising a unit dosage of a dry powder of tamsulosin or a pharmaceutically acceptable salt thereof in a controlled release matrix and to methods of making and using such formulation. The controlled release formulation is beneficial in the treatment of benign prostatic hyperplasia (BPH), particularly in those patients suffering from dysphagia. The controlled release formulation is easily dispersed in water or other suitable liquid, and so solves the problem of dysphagia, thereby improving patient compliance in that targeted patient population, yet the controlled release formulation has a release profile in a patient on an empty stomach similar to FLOMAX® ((R)-5-(2-{[2-(2-Ethoxyphenoxy)ethyl]amino}propyl)-2-methoxybenzene-1-sulfonamide hydrochloride) taken 30 minutes after a meal, but does not exhibit the FLOMAX® tablet food effect, thereby improving dosage form administration flexibility.

### PRIORITY CLAIM

This application claims priority of U.S. Provisional Application No. 62/331,599, filed May 4, 2016, the entire contents of which are incorporated herein by reference.

### BACKGROUND OF THE INVENTION

### 1. Field of the Invention

The present invention relates in one embodiment to formulations of tamsulosin hydrochloride, and, more specifically, to oral controlled release formulations of tamsulosin hydrochloride, and, especially, to oral controlled release suspension formulations of tamsulosin hydrochloride for patients that suffer from dysphagia.

### 2. Description of Related Art

Tamsulosin is an alpha1 adrenoreceptor antagonist that is approved by the FDA for the treatment of signs and symptoms of benign prostatic hyperplasia (BPH). The approved product, which is marketed in the United States as FLOMAX® ((R)-5-(2-{[2-(2-Ethoxyphenoxy)ethyl]amino}propyl)-2-methoxybenzene-1-sulfonamide hydrochloride), is an oral capsule (extended release) containing 0.4 mg of tamsulosin hydrochloride. The dosage and administration section of the approved prescribing information states that the capsules should not be crushed, chewed or opened.

Benign prostatic hyperplasia (BPH) is a condition where the stromal and epithelial cells in the prostate are enlarged. As the hyperplasia worsens, the urethra is impinged resulting in increased resistance to the flow of urine through the urethra. This results in incomplete voiding of urine, difficulty in urination, and may lead to atrophy of the muscle in the bladder wall, and to instability and weakness of the bladder wall. BPH is a common condition in men with the incidence and prevalence increasing with age.

There is a subset of the population that cannot swallow pills (capsules and tablets). The inability to swallow or difficulty swallowing is called dysphagia. Dysphagia may be a result of choking or fear of choking or may be due to comorbidities such as dry throat, blockage in the mouth or throat, and a number of other conditions and diseases. The comorbidities leading to dysphagia cause the incidence of dysphagia in the elderly to be increased, but the condition is not limited to the elderly and may afflict people of any age.

Currently, tamsulosin is only available in capsule form (extended release) and there are express instructions not to crush, chew, or open the capsule in the prescribing information of the approved products. Therefore, people that suffer from dysphagia that require tamsulosin for the treatment of signs and symptoms of benign prostatic hyperplasia will find no treatment or formulation option that will allow them to take tamsulosin.

Tamsulosin was first described in U.S. Pat. No. 4,703,063.

Controlled release tamsulosin formulations are also described in the prior art. For example, U.S. Pat. No. 4,772,475 describes a controlled released formulation of tamsulosin, microcrystalline cellulose and a release controlling agent, for example, a (meth)acrylic acid copolymer, such as EUDRAGIT® (methacrylate copolymer), granulated and formed into tablets and capsules. U.S. Pat. No. 7,018,658 describes a similar product, the distinction being the granules of the '658 patent comprise a core of tamsulosin that is coated with a shell of the release controlling agent. According to the '658 patent, the release profile of the dosage form of the '475 patent is not sufficient for an extended release dosage form (see the '658 patent at col. 2, ll 2-12), and a pellet inner core surrounded by an outer shell of a release controlling agent provides effective extended release (see the '658 patent at col. 3, ll 5-25.) Fleeting mention is made to “sachets” (see the '658 patent at col. 6, ll 14-16), but no details are provided how the sachets might be constructed, or how the granules might be administered in this form. Thus, capsules are themselves sometimes packaged in single-dosage sachets and this may be what is meant in view of the later discussion in the '658 patent at col. 6, ll 30-39, of a suitable package comprising a unit dosage amount of tamsulosin, including blister packs and plastic or glass bottles. There is no indication at any point that the tamsulosin is administered in the form of a liquid or that such liquid is the result of suspending a tamsulosin powder in a precursor liquid. Indeed, all examples relate to coated pellets.

A suspension formulation of tamsulosin that is not a tablet or capsule and can be more easily swallowed will provide patients that suffer from dysphagia with a formulation option that will allow them to take tamsulosin and receive the medication that they need.

FLOMAX® also exhibits distinct pH-dependent drug release. Administration to humans before food intake results in a 30% increase of bioavailability and a 40-70% increase in Cmax as compared to after food intake (Physicians Desk Reference 2002). As a result, labeling on FLOMAX® directs the tablets be taken approximately one-half hour following the same meal each day.

A formulation of tamsulosin that is free of these mealtime (“food effect”) constraints should increase patient compliance and afford administration flexibility.

Tamsulosin hydrochloride is also available in some countries as HARNAL® OCAS ((R)-5-(2-{[2-(2-Ethoxyphenoxy)ethyl]amino}propyl)-2-methoxybenzene-1-sulfonamide hydrochloride oral controlled absorption system). HARNAL® OCAS is a film-coated, prolonged release tablet containing macrogol 7,000,000, macrogol 8,000, magnesium stearate, butylated hydroxytoluene, colloidal silica anhydrous, hypromellose, and iron oxide yellow. HARNAL® OCAS tablets are purported to be only slightly affected by food and in a manner that is unlikely to be clinically significant.

HARNAL® OCAS tablets present the same challenges as FLOMAX® tablets for men suffering dysphagia.

### SUMMARY OF THE INVENTION

Certain embodiments of the present invention relate to a pharmaceutical dosage form comprising either tamsulosin freebase or a pharmaceutically acceptable salt thereof, especially tamsulosin hydrochloride, provided in a controlled release matrix, for example, in the form of a sachet, which pharmaceutical dosage form when mixed with water or other suitable liquid is drinkable, for patients with dysphagia. In one embodiment of the present invention tamsulosin hydrochloride is provided as an extended release suspension for easy administration.

As used herein, the term “controlled release” refers to the release of an agent such as a drug from a composition or dosage form in which the agent is released according to a desired profile over an extended period of time. Controlled release profiles include, for example, extended release, sustained release, prolonged release, pulsatile release, and delayed release profiles.

The present invention also relates in certain embodiments to a controlled release formulation comprising a sachet comprising a unit dosage of a dry powder of tamsulosin or a pharmaceutically acceptable salt thereof in a controlled release matrix.

The present invention further relates in certain embodiments to a method of treating benign prostatic hyperplasia in a patient suffering therefrom, the method comprising (A) providing a controlled release formulation according to the invention, (B) dispersing said formulation in a liquid to disperse the powder and thereafter (C) orally ingesting the liquid comprising the powder so dispersed.

The present invention further relates in certain embodiments to a method for preparing a controlled release formulation according to the invention comprising (A) preparing a dry powder of tamsulosin or a pharmaceutically acceptable salt thereof in a controlled release matrix and (B) weighing the powder into a sachet.

The present invention further relates in certain embodiments to a drinkable composition comprising (A) a liquid and (B) a dry powder of tamsulosin or a pharmaceutically acceptable salt thereof in a controlled release matrix dispersed therein.

### BRIEF DESCRIPTION OF THE DRAWINGS

The invention will now be described in greater detail with reference to the drawings, wherein:

FIG. 1 is a schematic of an individual particle according to the present invention.

FIGS. 2A, 2B and 2C are a series of prophetic graphs each depicting the percentage drug release as a function of time of an FDA-approved dosage form currently approved only in capsule or tablet form (squares) versus one dosage form embodiment according to the invention (triangles). FIG. 2A compares the release profile of the inventive dosage form with the release profile of a first combination of particles (A). FIG. 2B compares the release profiles when in the inventive dosage form the first combination of particles is intentionally supplemented with additional particles (B) to form a second combination of particles (A+B). FIG. 2C compares the release profiles when in the inventive dosage form the second combination of particles is intentionally supplemented with additional particles (C) to form a third combination of particles (A+B+C).

FIG. 3 compares the in vitro dissolution of an embodiment of the present invention to FLOMAX®.

FIG. 4 compares the in vivo bioequivalence of an embodiment of the present invention (“fasted” and “fed”) to FLOMAX® (“fasted” and “fed”).

### DETAILED DESCRIPTION OF THE INVENTION

Exemplary embodiments of the invention relate to formulations containing tamsulosin or a pharmaceutically acceptable salt thereof, particularly tamsulosin hydrochloride or another pharmaceutically acceptable salt thereof, designed to provide an extended release oral dosage form that can be easily swallowed by patients with dysphagia. Embodiments of the invention accomplish this with the attributes of the active pharmaceutical ingredient (API) and formulation components, carefully chosen for their contribution to controlled release oral suspension dosage forms. Embodiments of the invention in which a controlled release capsule or tablet dosage form has been replaced with a controlled release suspension offers the advantage that a patient with dysphagia can swallow it. This allows for treatment of a patient that otherwise would not be able to ingest a tablet or capsule. The composition of one exemplary embodiment of the present invention is comprised of a controlled release powder in a unit dose sachet, containing 0.4 mg tamsulosin hydrochloride, the known therapeutic daily dose for BPH. The composition of another exemplary embodiment of the present invention is comprised of an extended release suspension of 0.4 mg tamsulosin hydrochloride dispersed in a glass of a suitable liquid, especially water.

In one embodiment, the present invention relates to a pharmaceutical dosage form comprising an amount effective to treat benign prostatic hyperplasia of a combination of a plurality of particles of at least one mixture of (A) tamsulosin or a physiologically acceptable salt thereof in (B) a controlled release matrix, wherein the combination, when introduced to a quantity of liquid that is thereafter ingested by a patient on an empty stomach, exhibits a controlled release profile over time in the patient substantially identical to FLOMAX® ((R)-5-(2-{[2-(2-Ethoxyphenoxy)ethyl]amino}propyl)-2-methoxybenzene-1-sulfonamide hydrochloride) controlled release tablets or capsules ingested 30 minutes after a meal.

The pharmaceutical dosage form can be in the form of a sachet, as discussed, but can also be in the form of a capsule that can be dissolved in the liquid, for example, a water-soluble capsule, or is breakable or separable into two or more pieces to allow the powdery contents to be poured into the liquid. It is also possible to form a tablet by direct compression of the combination of particles. Although such a tablet, if sufficiently large in size, will not avoid dysphagia, such a tablet should avoid the FLOMAX® food effect.

The amount of tamsulosin or pharmaceutically acceptable salt thereof can be varied within a wide range, but is most preferably an effective amount to treat BPH, especially a unit dosage of 0.4 mg. Typically, the patient will be administered a single unit dosage of 0.4 mg daily, although a second unit dosage (for a total of 0.8 mg daily) can be administered if needed. Contemplated also is a dosage of tamsulosin hydrochloride ranging from 0.1 to 1 mg, preferably 0.4-0.8 mg, for the sachet and the other dosage forms described herein.

By “pharmaceutically acceptable salt” is meant all such salts as are conventional in pharmaceutical chemistry and formulation. As used herein, “pharmaceutically acceptable salts” refer to derivatives of the disclosed compounds wherein the parent compound is modified by making salts thereof. Pharmaceutically acceptable salts include salts of acidic (e.g., a carboxylic acid) or basic groups (e.g., a primary, secondary or tertiary amine) present in compounds disclosed herein. Examples of pharmaceutically acceptable salts include, but are not limited to, mineral or organic acid salts of basic residues such as amines; alkali or organic salts of acidic residues such as carboxylic acids; and the like.

The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, lauric, capric, myristic, palmitic, stearic, oleic, linoleic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic acids.

The pharmaceutically acceptable salts of the compounds can be synthesized from the parent compound (e.g., the unprotonated base form of the compound, often referred to as the “free base” of the compound), which contains a basic or acidic moiety, by conventional chemical methods. Generally, such salts can be prepared by reacting the free acid or base forms of these compounds with the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704, the disclosure of which is hereby incorporated by reference.

Pharmaceutically acceptable salts may also be prepared by reacting the free acid or base forms of compounds with an appropriate base or acid, respectively, in a melt process, optionally in the presence of other pharmaceutically acceptable excipients (e.g., waxes). As used herein, the term “melt process” refers to a process where the free acid or base forms of the compounds are dissolved in one or more excipients that are in molten form (i.e., it is a solid at room temperature) to make a solution wherein the base or acid interacts with the free acid or base form of the compounds, respectively, to form the desired pharmaceutically acceptable salt.

Other salts of the pharmaceutically active agent, which are contemplated by the present invention in order to alter the solubility and/or dissolution rate relative to the parent drug compound (e.g., the free acid or free base form of the compound) include, but are not limited to, pectinate, tannate, phytate, salicylate, saccharinate, acesulfamate, gallate, and terephthalate salts.

A significant side effect of tamsulosin (and other drugs) is that it leaves a bitter taste in the mouth, which is problematic when contemplating a liquid formulation to be swallowed. Accordingly, a preferred embodiment of the present invention involves formulating tamsulosin as the saccharinate (or other sugar salt). We contemplate the specific use of tamsulosin saccharinate as the active ingredient in any embodiment otherwise described herein. For example, if a specific example relates to tamsulosin hydrochloride, then the analogous embodiment wherein tamsulosin saccharinate is substituted for tamsulosin hydrochloride is likewise set forth by the effect of this paragraph.

Alternatively or in addition to the use of a sugar salt, flavoring agents and/or sweeteners may be utilized. Flavoring agents that may be used in the present invention include, and are not limited to, natural flavors, natural fruit flavors, artificial flavors, artificial fruit flavors, flavor enhancers or mixtures thereof. Natural flavors, artificial flavors or mixtures thereof include, and are not limited to, mint (e.g., peppermint or spearmint), lemon, lime, orange, strawberry, menthol, cinnamon, vanilla, artificial vanilla, chocolate, artificial chocolate or bubblegum. Natural fruit flavors, artificial fruit flavors or mixtures thereof include, and are not limited to, cherry, grape, orange, strawberry or lemon. Flavor enhancers include, and are not limited to, citric acid. Although flavoring agents are generally provided as a minor component and in amounts effective to provide a palatable flavor to the liquid pharmaceutical composition, the addition of at least one flavoring agent is preferred; and, more preferably, up to two flavoring agents may be employed. A flavoring agent used in the inventive formulations in a range of from about 0.01 to about 0.15 grams per 100 mL. The flavorings are generally utilized in amounts that will vary depending upon the individual flavor, and may, for example, range in amounts of about 0.01% to about 10% by weight/weight of the final composition of the sachet.

Examples of sweeteners include sweetening agents, artificial sweeteners and dipeptide based sweeteners, e.g., monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, mannitol, saccharin salts, i.e., sodium, or calcium saccharin salts, cyclamate salts, acesulfam-K, ammonium glycyrrhizinate, dipotassium glycyrrhizinate and the free acid form of saccharin L-aspartylphenylalanine methyl ester and mixtures thereof.

Generally, the sweetener will be present in an amount corresponding to about 1 to 60% weight/weight of the total composition of the sachet, the amount depending in part upon whether other sweetener ingredients are present and the level of sweetness desired. Typically when sugar is used, it is present from about 10% to about 50% w/v of the composition. It will be appreciated that combinations of sweeteners can be used. The sweetening agents, when used, may also be used alone or in combination with each other. When an artificial sweetness enhancer is used it may be present in an amount from about 0.05% to about 15% weight/weight of the final composition of the sachet.

Other additives include surfactants, for example, sodium dodecyl sulfate (sodium lauryl sulfate), sorbitan laurate, polyacrylate and salts thereof, for example, sodium polyacrylate, sodium diocotyl sulfosuccinate, sodium oxylate, sodium tartrate, and mixtures thereof, or other surfactants well known to persons skilled in the art; or diluents, for example, hydroxypropyl methyl cellulose, cellulose, talc, starches, for example, corn starch, rice starch, tapioca, wheat starch, potato starch, pre-gelatinized starch, or sodium starch glycolate, or gelatin, and mixtures thereof, or other diluents well known to persons skilled in the art.

In certain embodiments, the tamsulosin or pharmaceutically acceptable salt thereof is provided in a controlled release formulation in the form of a dry powder for dispersion in a suitable liquid, for example, water, fruit juices (orange juice, apple juice, pineapple juice, etc.). Typically, flavorings, sweeteners, surfactants, diluents and other conventional excipients will be combined in the controlled release formulation as powders themselves.

The tamsulosin or pharmaceutically acceptable salt is provided in a controlled release matrix that is compatible with the API, is in a form that facilitates suspension of the controlled release formulation in the liquid and slowly releases the API once the liquid containing the suspended controlled release formulation is ingested by the patient.

In an especially preferred embodiment, the controlled release formulation is contained in a sachet. The API and the controlled release matrix will be mixed, preferably, homogeneously, and reduced to a dry powder, preferably by extruding the mixture as an extrudate and thereafter crushing or, preferably, micronizing. The resulting dry powder is then weighed and packaged into sachets, if desired, along with the flavorings, sweeteners, surfactants, diluents and other conventional excipients. The weight is selected so that, for example, the unit dosage of 0.4 mg of tamsulosin or pharmaceutically acceptable salt is provided per sachet.

Patients are given the sachets with instructions to disperse one sachet each day into a suitable liquid for treatment of BPH (or other disease or disorder). The patient will disperse the sachet into a suitable liquid of their choice, for example, water or fruit juice, and then ingest the resulting suspension. Since the suspension will be easier for patients suffering dysphagia to tolerate, patient compliance should increase over the rates currently observed with tamsulosin capsules.

Further, since the release from the inventive suspension (or capsules or tablets) does not increase dramatically upon taking proximate to a meal, as is the case with FLOMAX®, the inventive suspension (or capsules or tablets) can be taken on an empty stomach, for example, in the morning, or at bedtime, thereby, also increasing patient compliance and administration flexibility. Indeed, the effect of food on the inventive dosage form is fundamentally different than the effect of food on FLOMAX®. While food intake slows the release of the active ingredient from the inventive dosage forms, in stark contrast to the situation with FLOMAX®, wherein the release rate increases in the absence of food, the slowing of the release rate in the case of the inventive dosage forms does not constitute a safety concern.

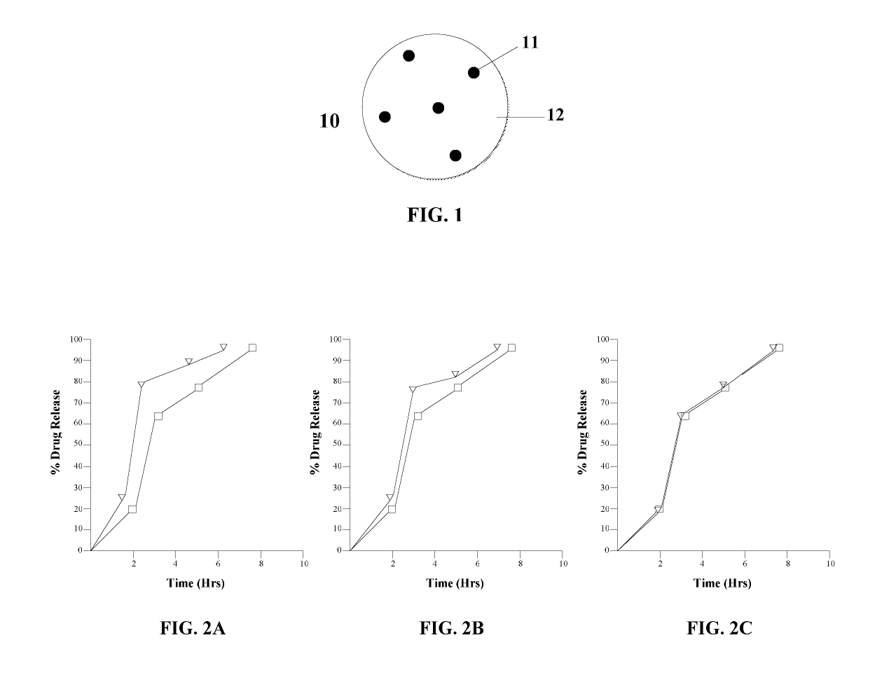

The dry powder of the API and the controlled release matrix will comprise a plurality of particles selected and combined in such a way that the pharmaceutical dosage form when introduced to a quantity of liquid that is thereafter ingested by a patient exhibits a desired controlled release profile of the API in the patient over time. Referring to FIG. 1, there is shown an illustration of one embodiment of a particle 10 according to the present invention. The particle comprises a controlled release matrix 12, throughout which are distributed, preferably homogeneously, molecules of API 11. When these particles are ingested into the patient's body along with the suspending liquid, the API molecules 11 will be released from the controlled release matrix 12.

The factors influencing drug release from matrix materials are well known in the art. Reference is made, for example, to M. Varma et al., “Factors Affecting Mechanism and Kinetics of Drug Release from Matrix-Based Oral Controlled Drug Delivery Systems,” Am. J. Drug Deliv., 2(1): 43-57 (2004), the contents of which are incorporated herein by reference. The present invention makes use of the fact that differences in terms of API release rate exist between different particles produced from the same mixture of API+matrix, or between different particles produced from different mixtures of API+matrix, to produce a composite grouping of particles, intentionally individually selected on the basis of their differences in releasing of the API, that collectively combine to release the API in the desired controlled API release rate. In one preferred embodiment, the selection can be as simple as separating off unusually small particles and/or unusually large particles, for example, with the aid of one or more mesh screens, until a combination of particles is obtained that, when combined with other ingredients of the dosage form, for example, sweetners and other excipients, mimics the release of FLOMAX® in a patient. In another preferred embodiment, individual particles are generated, tested for their release properties alone and in combination with expected co-contents of the sachet, the liquid and in the body and the results are noted and catalogued. Various types of individual particles so catalogued are selected and combined in a way that is calculated to yield the desired release profile, and then this calculation is confirmed by actual testing. Selection and combining individual particles in this way is illustrated in FIGS. 2A-2C.

FIG. 2A shows one embodiment of the inventive dosage form with a first combination of particles (A). This first combination of particles (A) may give a release profile (triangles) that is significantly different from that exhibited by the FDA-approved dosage form (squares) under the identical testing conditions. The invention contemplates in this particular embodiment modifying this first combination of particles (A) by intentionally supplementing with additional particles (B) to form a second combination of particles (A+B) that exhibits a release profile that is in closer agreement to that exhibited by the FDA-approved dosage form, as shown in FIG. 2B. Further modifications can be made in the same way until the two release profiles are substantially identical as shown in FIG. 2C. By “substantially identical” is meant at least 80% agreement between the two release profiles, preferably at least 90% agreement, more preferably at least 95% agreement, most preferably at least 99% agreement.

In one embodiment, the pharmaceutical dosage form according to the invention comprises a plurality of particles in a size distribution selected such that the desired release rate over time is achieved when the pharmaceutical dosage form is introduced to the quantity of liquid and ingested by the patient.

In another embodiment, the pharmaceutical dosage form according to the invention comprises a plurality of particles being selected from different mixtures of API and controlled release matrix and combined such that the desired release rate over time is achieved when the pharmaceutical dosage form is introduced to the quantity of liquid and ingested by the patient. In one especially preferred embodiment, the pharmaceutical dosage form comprises particles of API in a first controlled matrix and also particles of API in a second controlled matrix, wherein the first controlled matrix and the second controlled matrix are different. In a second especially preferred embodiment, the pharmaceutical dosage form comprises particles of a first concentration of API in a first controlled matrix and also particles of a second concentration of API in a second controlled matrix, wherein the first concentration of API is different from the second concentration of API, and wherein the first controlled matrix is identical to or different from said second controlled matrix.

The pharmaceutical formulations according to the invention do not per se comprise any enteric coating that dissolves as a function of pH. These coatings, which are well known to persons skilled in the art, allow a dosage form even though ingested to pass through the stomach without liberating the active ingredient, which then only or primarily undergoes controlled release in the intestinal tract. Such enteric coatings preferably dissolve at a pH of between 5 and 7.

Although the inventive dosage form does not contain such an enteric coating per see, it may be advantageous to combine enteric coating materials in the controlled release matrix.

These enteric coating materials are preferably selected from the group of shellac, polymethacrylic acid/ethyl acrylate or methacrylic acid/methyl acrylate/methyl methacrylate copolymer, methacrylic acid/methyl methacrylate copolymers, hydroxypropyl methyl cellulose acetate-succinate, cellulose acetate-phthalate, polyvinyl acetate-phthalate, hydroxypropyl methyl cellulose phthalate and/or cellulose acetate-trimellitate.

Further retardation of release over and above that caused in situ, and hence a further modification of the release of the active ingredient can be effected by a variety of methods known to those skilled in the art.

Matrix materials which can be used for instant invention include physiologically compatible, hydrophilic materials known to those skilled in the art, e.g. Bauer, et al., “berzogene Arzneiformen” (“Coated Pharmaceutical Forms”), Wissenschaftliche Verlagsgesellschaft mbH Stuttgart, 1988, p. 69 et seq., which is incorporated herein by reference and thus forms part of this disclosure. The hydrophilic matrix materials used are preferably polymers and particularly preferably cellulose ethers, cellulose esters and/or acrylic resins, preferably poly(meth)acrylates. The matrix materials used are very particularly preferably ethyl cellulose, hydroxypropyl methyl cellulose, hydroxypropyl cellulose, hydroxymethyl cellulose, poly(meth)acrylic acid and/or derivatives thereof such as their salts, amides or esters.

Other preferred matrix materials include hydrophobic materials such as hydrophobic polymers, waxes, fats, long-chain fatty acids, fatty alcohols or corresponding esters or ethers, or mixtures thereof. The hydrophobic materials used are particularly preferably C₁₂-C₃₀ fatty acid mono- or diglycerides and/or C₁₂-C₃₀ fatty alcohols and/or waxes, or mixtures thereof.

Materials suitable for the sustained-release matrix also include polyalkylene oxides, preferably, polyethylene oxide; polyvinyl acetates; and polyvinyl pyrrolidones.

The sustained-release matrix material used can also be mixtures of said hydrophilic and hydrophobic materials.

The matrix components will make up the bulk of the inventive dosage form. For a unit dosage form containing the preferred 0.4 mg of tamsulosin, the amount of the matrix components will total from 50-1000 mg, preferably 75-500 mg, most preferably 100-400 mg.

To adjust the rate of release of the active substances, the inventive formulations can optionally contain, in addition to the water-insoluble polymers, non-retarding, preferably water-soluble polymers in amounts of up to 30 wt. %, such as polyvinylpyrrolidone; or water-soluble celluloses, preferably hydroxypropyl methyl cellulose or hydroxypropyl cellulose; and/or hydrophilic pore-forming agents such as sucrose, sodium chloride or mannitol; and/or plasticizers known in the art.

To further retard the release of the active ingredient, the pharmaceutical formulations according to the invention can preferably also contain the active ingredient uniformly distributed in a sustained-release matrix.

In a preferred embodiment, the controlled release material comprises at least one material selected from the group consisting of ethyl cellulose, hydroxypropyl methyl cellulose, and derivatives thereof.

In a more preferred embodiment, the controlled release material comprises a mixture of (i) ethyl cellulose or a derivative thereof, and (ii) hydroxypropyl methyl cellulose or a derivative thereof.

In a most preferred embodiment, the ethyl cellulose or derivative thereof is selected from the group consisting of ethyl cellulose, and blends of ethyl cellulose and cellulose acetate phthalate; and the hydroxypropyl methyl cellulose or derivative thereof is selected from the group consisting of hydroxypropyl methyl cellulose, hydroxypropyl methyl cellulose acetate succinate, and hydroxypropyl methyl cellulose phthalate.

Particle size and matrix composition are factors influencing the release profile of the active ingredient from the inventive formulations. Particle sizes range from 1-1000 μm, especially, 1-500 μm, preferably <10-400 μm, most preferably <10-350 μm. Smaller sized particles within these ranges may improve the aesthetics of the dispersed products, for example, as explained in U.S. Pat. No. 5,149,541, which pertains to METAMUCIL® (psyllium husk). A combination of particles ranging in size between 100-600 μm, preferably 125-500 μm, most preferably 150-450 μm provides especially good results where the matrix is based on cellulose derivatives, particularly ethyl cellulose and/or hydroxypropyl methyl cellulose, and acid esters thereof.

In a most preferred preparation embodiment, the inventive dosage form is prepared by (A) extruding an extrudate comprising: a mixture of (i) tamsulosin or a salt thereof, (ii) hydroxypropyl methyl cellulose or a derivative thereof, and (iii) ethyl cellulose or a derivative thereof, (B) reducing the extrudate to a powder, (C) selecting from said powder particles of various sizes to give a combination of selected particles exhibiting a distribution of particle sizes such that when the combination of selected particles is ingested by a patient on an empty stomach along with a quantity of liquid and optionally excipients there is produced in said patient an in vivo release profile of tamsulosin or salt thereof over the course of a given period of time that substantially mimics the in vivo release profile over the same period of time of FLOMAX® ingested by said patient 30 minutes after a meal.

A brief scheme for adapting any suitable active ingredient to the present invention is shown below for an extruded product.

### Brief Scheme:

### Development Steps for Particulate Component of Sachet:

Establish the matrix compatibilities with tamsulosin HCl
First Tier Alternative matrix components
   50 to 80% VA64 (vinylpyrrolidone-vinyl acetate)
   2 to 10% PEG1500 (polyethylene glycol 500)/40 to
   90% VA64/10% to 40% HPMCs
   10% PEG1500/40% VA64
   10% TPGS (Tocopherol propylene glycol succinate)/40 to 10%
   TPGS/40% VA64
   HPMCAS MF (hydroxypropylmethylcellulose acetate-succinate)
   Eudragit L30
   Eudragit RS PO
   HPMCT (hydroxypropylmethylcellulose trimellitate)
Second Tier Alternative matrix components
   Methacrylic acid copolymer type A, NF
   Methacrylic acid copolymer type B, NF
   Methacrylic acid copolymer type C, NF
   Polyethylene oxide
   Polyvinylpyrrolidone
   Ethylene vinyl acetate
   Poly(L-lactic-co-glucolic acid)
   Polycaprolate
   Microcrystalline wax
   Xanthum gum
   Propylene glycol
   Ethyl cellulose
   Glycerol distearate
   Glycerol tristearate
   Glycerol dioleate
   Glycerol trioleate
Matrix Diluents
   Docusate sodium
   Xanthum gum
   Polysorbate 80
   Microcrystalline cellulose
   Polyethylene glycols
Matrix Lubricants
   Stearic acid
   Sodium lauryl sulfate
   Starch
   Pregelatinized starch
   talc

The mixture of matrix components will be extruded or spheronized.
Prepare the particulates in size ranges:
   generate separate milled particle fractions;
      of <10 micron,
      10 to 20 micron,
      20 to 30 micron,
      30 to 40 micron,
      40 to 50 micron,
      50 to 60 micron and
      60 to 70 micron.
Blend the particle size fractions to establish an appropriate particle size mixture

### Development of Sachet Mixture:

Add surfactant, diluent powder, and sweetener
Surfactants and/or dispersing agent
   Sodium dodecyl sulfate (Sodium lauryl sulfate)
   Sorbitan laurate
   Polyacrylate
   Sodium polyacrylate
   Sodium diocotyl sulfosuccinate
   Sodium oxylate
   Sodium tartrate
Diluent
   HPMC
   Cellulose
   Talc
   Corn Starch
   Rice Starch
   Tapioca
   Wheat starch
   Potato starch
   Pre-gelatinized starch
   Sodium starch glycolate
   Gelatin
Flavorings (GRAS agents)
   Mannitol
   Sorbitol
   Strawberry flavor
   Lemon flavor
   Lime flavor
   Sucralose

Blend the surfactant, diluent powder and sweetener

### Characterize the Sachet in a Diluent for Drinking

Prepare various slurries compatible with sachet components;
Slurry in 8 oz water
Slurry in 8 oz juice
   Orange
   Cranberry
   Tomato
   grapefruit
Slurry in 8 oz apple sauce

Characterize per target product profile (See Table 1)
Potency
Drug release profile
Impurities

TABLE 1
Development Target Product Profile for Powder for Oral Suspension

| DTPP Parameter | Target |
|---|---|
| Name | Tamsulosin HCl Sachet |
| API | tamsulosin HCl |
| Dosage Strengths | 0.4 mg, once daily |
| Target Patient | Adult male |
| Population | |
| Pediatric or | Not for female use, not indicated for pediatric use. No overall |
| Gender Use | differences in efficacy or safety |
| Indication | Tamsulosin is an alpha₁ adrenoreceptor indicated for treatment |
| | of the signs and symptoms of benign prostatic hyperplasia |
| Schedule Class | N/A |
| Dosage | Sachets (~200 mg or less), single use pouch |
| | “Reconstitute in water or juice (8 oz) and drink” to be modified |
| | as appropriate |
| Container closure | Aluminum pouch |
| system | |
| Storage | Store at Controlled Room Temperature encompassing 20°-25° C. |
| | (68° to 77° F.), allowing for excursions between 15°-30° C. |
| | (59° F.-86° F.) per USP General Notices |
| Appearance | Free flowing particles |
| Potency | Release: 90.0% to 110.0% |
| | Stability: 90.0% to 110.0% |
| Uniformity of | Complies with requirements of USP <905> |
| Dosage Units | |
| Dissolution | Complies with USP <711> dissolution testing requirements for |
| | tamsulosin (capsule USP monograph Test 3); 8 hr total test |
| | time |
| | Acid stage: 2 hr in 0.003% polysorbate 80 pH 1.2 (500 mL) |
| | 100 rpm |
| | Buffer stage: 6 hr total, phosphate buffer pH 7.2 |
| | At 2 hr; NMT 20% |
| | At 3 hr; 50% to 85% dissolved |
| | At 8 hr: NLT 80% dissolved |
| | (See USP tamuslosin capsule test 3 for method description) |
| Impurities | Complies with ICH and/or FDA requirements for impurities in |
| | drug products |
| Residual Solvents | Controlled per USP <467> per each supplier's statement for |
| | components. None added during processing for drug product |
| Microbial | Meets USP acceptance criteria for non-aqueous preparations |
| evaluation | for oral use |
| Stability | Shelf life ≥ 12 months, prefer 24 to 36 months |

DTPP = development target product profile

### Drug Release Method

Use USP method for tamsulosin HCl tablets.
Compare experimental formulation with LD in various media
   Stage 1: 2 hours in acidic buffer: 0.003% polysorbate 80 in pH 1.2 (using HCl)
      Use Apparatus 2 for tablets (for reference), no chamber required for sachet
      100 rpm
      Test time 0, 30 min, 1 h, 1.5 h, and 2 h
   Followed by Stage 2: dissolution profile in phosphate buffer pH 7.2
      Use Apparatus 2 for tablets (for reference) no chamber required for sachet
      100 rpm
      Test 0, 1 h, 2 h, 3 h, 4 h, 5 h, and 6 h
   Additional dissolution profiles generated in pH 1.2, 4.5 and 6.8 medium per the SUPAC-MR Guidance requirements Equivalency will be established by achieving multi-point dissolution similarity (f2) values of >50 with results obtained for the experimental formulation and the LD The optimum formulation will show dissolution similarity (f2) values of >50 when compared to the LD dissolution profile

The invention will now be described in greater detail with reference to the following non-limiting examples.

### EXAMPLES

### Example 1: (Prophetic)

The following components, in the percents by weight indicated, are homogeneously mixed in a mixer, for example a roller mixer, a shaking mixer, a shearing mixer or a compulsory mixer:

| | |
|---|---|
| Tamsulosin hydrochloride | 0.1-10% |
| Polyethylene glycol | 30-80% |
| Sodium lauryl sulfate | 1-10% |
| Pregelatinized starch | 1-5% |
| Microcrystalline cellulose | 5-20% |

(Percents given are by weight, based on the total weight of the formulation.)

The mixture is extruded through a twin screw extruder, for example, the twin screw extruder available from the company Leistritz (Nurnberg) of type ZSE 18 HP 40D, preferably with screws equipped with eccentric screw ends. A heatable nozzle plate with 8 orifices each having a diameter of 1.0 mm can be used as the nozzle. The extrusion parameters can be set, for example at the following values: screw speed: 150 rpm; throughput: 2 kg/h; product temperature: 60° C. to 140° C., preferably 80° C. to 140° C., most preferably 110° C. to 140° C. with corresponding barrel temperature.

The rod-shaped extrudate is cut at predetermined lengths and the cut pieces are reduced to powder by micronization in suitable equipment. The powder particles are separated according to size and, based on knowledge of the release properties of individual particles, selected on this basis and recombined into a new powder expected to have a desired release profile of API.

The powder is weighed out and along with flavorings, surfactants and sweeteners filled into sachets each containing a unit dosage of 0.4 mg of tamsulosin hydrochloride.

### Example 2: (Prophetic)

The following components, in the percents by weight indicated, are homogeneously mixed in a mixer, extruded, cut and micronized in a manner analogous to Example 1:

| | |
|---|---|
| Tamsulosin hydrochloride | 0.1-10% |
| Hydroxymethyl propyl cellulose | 30-80% |
| Sodium lauryl sulfate | 1-10% |
| Pregelatinized starch | 1-5% |
| Microcrystalline cellulose | 5-20% |

The powder particles are separated according to size and, based on knowledge of the release properties of individual particles, selected on this basis and recombined along with desired particles from Example 1 into a new powder expected to have a desired release profile of API.

The powder is weighed out and along with flavorings, surfactants and sweeteners filled into sachets each containing a unit dosage of 0.4 mg of tamsulosin hydrochloride.

### Example 3: (Prophetic)

A crystalline active pharmaceutical ingredient extruded with a carrier and release controlling agent, milled and packaged in a sachet.

| | |
|---|---|
| Tamsulosin saccharinate, micronized | 0.1-10% |
| Polyethylene glycol | 30-80% |
| Sodium lauryl sulfate | 1-10% |
| Pregelatinized starch | 1-5% |
| Microcrystalline cellulose | 5-20% |
| Flavoring | 5-30% |

(Percents given are by weight, based on the total weight of the formulation.)

### Example 4: (Actual)

The following ingredients were combined as a pre-extrusion blend:

| | |
|---|---|
| Tamsulosin HCl | 10 wt. % |
| Ethocel Standard 10 Premium | 45 wt. % |
| Hypromellose Acetate Succinate, NF, AS- | 45 wt. % |
| MMP Grade | |

*All “wt. %” based on the total weight of the pre-extrusion blend

The pre-extrusion blend was introduced to the hopper and extruded through the die of a Leistritz (Nurnberg, Germany) ZSE 18 HP 40:1 Twin Screw extruder operating at a screw speed of 100±10 RPM; throughput: 0.5±0.2 kg/hr; melt plate temperature: 160° C.±5° C.; heating zone temperature: 160° C.±5° C.; and die heater temperature: 160° C.±5° C. The extrudate was extruded onto a conveyor belt and cooled. The cooled extrudate was milled using a Fitzmill with a 0.033″ perforated screen with the blades in the hammer position, until a powder was obtained. This powder was subjected to 40-mesh and 100-mesh screens to eliminate fines and coarse particles, and to yield a particle size range for the resulting powder between 150-425 μm.

A portion of this powder was then combined with dry excipients into a sachet having the following formulation:

| | % | mg/sachet |
|---|---|---|
| Tamsulosin HCl | 1.1 | 0.452 |
| Ethocel Standard 10 Premium | 5 | 2 |
| Hypromellose Acetate Succinate, NF, AS- | 5 | 2 |
| MMP Grade | | |
| Avicel | 85.9 | 34.8 |
| flavor | 1 | 0.4 |
| sucralose | 1 | 0.4 |
| sodium stearyl fumerate | 0.5 | 0.4 |
| silicon dioxide | 0.5 | 0.4 |
| Total | 100 | 40.512 |

### Example 5: (Prophetic)

The formulation of Example 4 is, instead of being combined into a sachet, combined into a water-soluble, two-part capsule. The contents can be introduced to a liquid, for example, water or juice, simply by dropping the entire capsule into the liquid, or by separating the two parts of the capsule and pouring the capsule contents into the liquid.

### Example 6: (Prophetic)

The formulation of Example 4 is, instead of being combined into a sachet, pressed on a tablet-press into a round or other-shaped pill. The pill can be swallowed in the same manner as FLOMAX® capsules. However, because the inventive capsules have a different composition, they should be free from the FLOMAX® capsule food effect.

### Example 7: (Actual): Dissolution Studies

### Objective

The primary objective of this study was to establish by HPLC the similarity of the in vitro profile of the sachet formulation of Example 4 compared to the in vitro profile of FLOMAX® capsules when measured according to USP <711> dissolution testing requirements for tamsulosin (capsule USP monograph Dissolution Test 3):

8 hr total test time;

Acid stage: 2 hr in 0.003% polysorbate 80 pH 1.2 (500 mL) 100 rpm;

Buffer stage: 6 hr total, phosphate buffer pH 7.2;

At 3 hr: 65% to 85% dissolved; and

At 8 hr: NLT 80% dissolved.

### Protocol:

### Dissolution Parameters

### USP Apparatus: USP Apparatus II (Paddle)

### Rotation Speed: 100 RPM

Sample Volume: 5 mL with no media replacement

### Sinkers/Baskets: None

Filters: 0.45 μm nylon

### Bath Temperature: 37° C.±0.5° C.

Pull Times: 2 hrs, 3 hrs & 8 hrs

### Acid Stage

500 mL of acid stage media was added to each dissolution vessel, and allowed to equilibrate to 37° C.±0.5° C. Each formulation was weighed and the whole contents removed and sprinkled carefully into each of six (6) dissolution vessels and the timer was started. At 2 hours, a single 5 mL sample was removed from each vessel using a cannula. The sample was taken from the zone midway between the surface of the dissolution medium and the top of the paddle blade and not less than 1 cm from the vessel wall and shaft. The sample was filtered directly to a glass HPLC vial using a 0.45 μm nylon membrane.

### Dissolution

500 mL of pre-warmed (to 37° C.) buffer stage concentrate was added into each of the six (6) dissolution vessels containing the acid stage media. The total volume was then 1000 mL and had a pH of 7.2. The timer was allowed to continue. At 3 hours and 8 hours, a single 5 mL sample from each vessel was removed using a cannula. The sample was taken from the zone midway between the surface of the dissolution medium and the top of the paddle blade and not less than 1 cm from the vessel wall and shaft. The sample was filtered directly to a glass HPLC vial using a 0.45 μm nylon membrane. The samples would expire in three (3) days stored at ambient laboratory conditions.

### HPLC Parameters

### Instrument: Waters Alliance™ 2695 HPLC System

Column: Atlantis, dC18, 5 μm, 4.6×150 mm
Flow Rate: 1.2 mL/min

### Run Time: 10 min

### Sample Temp: Ambient

### Column Temp: 40° C.

### Injection Volume: 100 μL

### Detection: 225 (nm)

### Pump: Isocratic

### Needle Wash/Seal Wash: Acetonitrile/Water (50/50)

### Results:

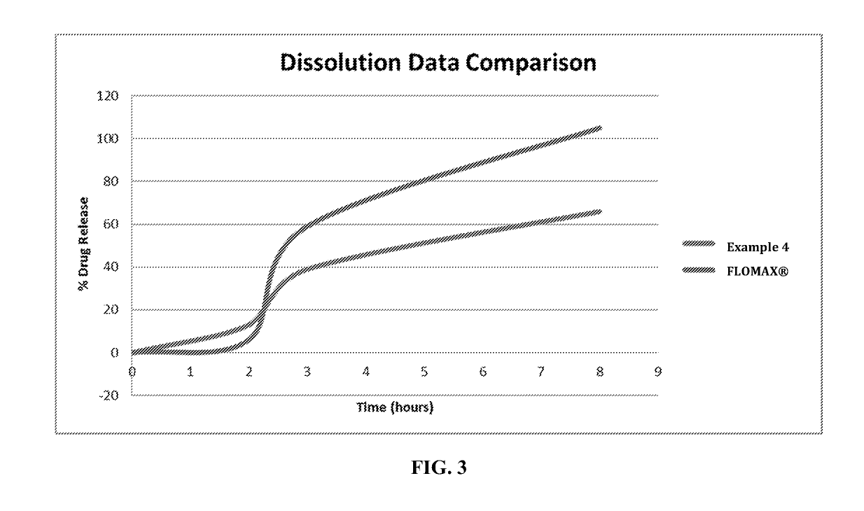

Dissolution of the formulation of Example 4 was compared to FLOMAX® capsules. The results are shown in FIG. 3. The two curves were sufficiently similar to justify further studies in vitro and in vivo.

### Example 8: (Actual): Clinical Studies

### Objective

The primary objective of this study was to establish the bioequivalence of the formulation of Example 4 (“Tamsulosin DRS” hereinbelow) to FLOMAX® capsules under both fasted and fed conditions. Secondary objectives included assessing the effect of food, or the lack thereof, on the pharmacokinetics (PK) of Tamsulosin DRS; and assessing the safety and tolerability of a single dose of Tamsulosin DRS with and without food.

The study was designed as a two-stage study. Potential subjects were screened for study eligibility within 30 days before the start of the study on Day 0/Period 1.

### Stage 1: Formulation Assessment and Dosing Scheme

Stage 1 was an open-label, partially randomized, three-period crossover design. Subjects were randomized into one of two sequences. A total of 12 subjects under fasted and fed conditions were enrolled in Stage 1. Dropouts were not replaced. The dosing scheme was as follows:

| | Period 1 | Period 2 | Period 3 |
|---|---|---|---|
| | Dosing Day | | |
| | Day 0 | Day 7 | Day 14 |
| Formulation 1A | FLOMAX ® | Tamsulosin DRS | Tamsulosin DRS |
| (n = 6) | (Fed) | (Fasting) | (Fed) |
| Formulation 1B | FLOMAX ® | Tamsulosin DRS | Tamsulosin DRS |
| (n = 6) | (Fed) | (Fed) | (Fasting) |

After Stage 1, bioanalytical analysis of the samples was conducted.

### Stage 2: Bioequivalence and Food Effect

Stage 2 was an open-label, randomized, single-center, single-dose, 4-period, crossover, bioavailability study. In each period, the subject received a single dose of either Tamsulosin DRS (test) or FLOMAX® (reference). A total of 36 subjects were enrolled in Stage 2. Dropouts were not replaced unless subject enrollment fell below 30 completed subjects. The dosing scheme was as follows:

| | Period 4 | Period 5 | Period 6 | Period 7 |
|---|---|---|---|---|
| | Dosing Day | | | |
| | Day 42 | Day 49 | Day 56 | Day 63 |
| Group 1 (n = 9) | Tamsulosin DRS | FLOMAX ® | Tamsulosin DRS | FLOMAX ® |
| | (Fasting) | (Fasting) | (Fed) | (Fed) |
| Group 2 (n = 9) | FLOMAX ® | Tamsulosin DRS | FLOMAX ® | Tamsulosin DRS |
| | (Fed) | (Fasting) | (Fasting) | (Fed) |
| Group 3 (n = 9) | Tamsulosin DRS | FLOMAX ® | Tamsulosin DRS | FLOMAX ® |
| | (Fed) | (Fed) | (Fasting) | (Fasting) |
| Group 4 (n = 9) | FLOMAX ® | Tamsulosin DRS | FLOMAX ® | Tamsulosin DRS |
| | (Fasting) | (Fed) | (Fed) | (Fasting) |

### Inclusion Criteria

Subjects had to fulfill all of the following inclusion criteria to be eligible for participation in the study unless otherwise specified:
1. Healthy adult male volunteers 18 and 60 years of age (inclusive).
2. Able to understand and provide signed informed consent.
3. Willing to comply with the requirements of the study.
4. Seated blood pressure between 100 and 140 mmHg systolic and between 60 and 90 mmHg diastolic (inclusive).
5. Seated pulse rate between 45 and 100 beats/minute (inclusive).
6. Normally active and otherwise judged to be in good health on the basis of medical history and physical examination.
7. Body mass index (BMI) 19.0 and 32.0 kg/m2.
8. Body weight>55 kg.

### Exclusion Criteria

Subjects were not enrolled in the study if they met any of the following exclusion criteria:
1. History of any clinically significant cardiovascular, hepatic, renal, pulmonary, hematologic, gastrointestinal, endocrine, immunologic, dermatologic, or neurologic disease.
2. Any use of prescription medications within 28 days prior to check-in for Period 1 for Stage 1 and Period 4 for Stage 2. Currently taking any medication for treatment of hypertension. Currently taking any medication for treatment of hypercholesterolemia.
3. Any use of over-the-counter (OTC) medicines within 7 days prior to check-in for Period 1 for Stage 1 and Period 4 for Stage 2.
4. History of allergic reaction to tamsulosin or other related drugs.
5. Abnormal and clinically relevant (as determined by the principal investigator [PI]) electrocardiogram (ECG) tracing.
6. Clinically significant illness or surgery within 28 days prior to dosing (including flu, flu-like symptoms, diarrhea, vomiting) or acute illness at the time of either the pre-study medical evaluation or dosing.
7. Use of any medication known to alter hepatic enzyme activity within 28 days prior to the initial dose of study medication (eg, omeprazole or other proton pump inhibitors [PPIs])
8. Positive test for hepatitis B, hepatitis C, or human immunodeficiency virus (HIV) at screening.
9. Any clinically significant abnormal laboratory test results found during medical screening as determined by the Investigator. The definition of clinically significant will be related to the normal levels of each test at the local laboratory conducting the test. NOTE: A test value above or below the normal range does not necessarily indicate that the value is “clinically significant.” The determination should be made at the discretion of the Investigator with consultation, when necessary, with the Medical Monitor.
10. Alanine aminotransferase (ALT) or aspartate aminotransferase (AST) levels >1.25 times the upper limit of normal at screening or check-in for Period 1.
11. A positive drug screen or continine screen.
12. History of use of any nicotine products in the 3 months preceding the study start (first dose).
13. A history of major mental illness that in the opinion of the Investigator may affect the ability of the subject to participate in the study. Institutionalized subjects will not be eligible for participation.
14. Exposure to any investigational agent within 30 days prior to study start (first dose).
15. Exposure to tamsulosin within 30 days prior to study start (first dose).
16. Subject has made a donation (standard donation amount or more) of blood or blood products (with the exception of plasma as noted below) within 56 days prior to the study start (first dose).
17. Subject has made a plasma donation within 7 days prior to the study start (first dose).
18. Subject has a condition the Investigator believes would interfere with the ability to provide informed consent or comply with study instructions, or that might confound the interpretation of the study results or put the subject at undue risk.

### Study Procedures:

### Screening

Potential subjects will be screened for study eligibility within 30 days before the start of the study on Day 0/Period 1 for Stage 1 and within 30 days of Day 42/Period 4 for Stage 2. Subjects will be instructed to fast for at least 8 hours (for laboratory profiles) before arriving for the screening visit. Screening evaluations will include the following:
6. Signed informed consent
7. Determination of eligibility, inclusion and exclusion criteria (as many as can be determined at the screening visit)
8. Medical history
9. Previous (used in the previous 30 days) and concomitant medication (currently using)
10. Physical examination, including height and weight without shoes
11. BMI determination
12. Vital signs (blood pressure, pulse, respirations, and temperature) after sitting for at least 3 minutes
13. 12-lead ECG, supine for at least 3 minutes
14. Laboratory profiles including hematology, chemistry panels, and urinalysis
15. HIV antibody, hepatitis B surface antigen, and antibody titer against hepatitis C
16. Toxicology screen for drugs of abuse and alcohol breathalyzer test.

Results must be negative for subjects to continue in the study.
17. Subjects will be instructed in the following restrictions:
   a. Stop the use of prescription medications within 28 days prior to check-in for Period 1 (Stage 1) or Period 4 (Stage 2).
   b. Stop the use of OTC medicines within 7 days prior to check-in for Period 1 (Stage 1) or Period 4 (Stage 2) (including any non-steroidal anti-inflammatory drugs [NSAIDs] and aspirin-containing medications).
   c. Stop the use of vitamins or herbal supplements within 7 days prior to check-in for Period 1 (Stage 1) or Period 4 (Stage 2).
   d. Abstain from foods containing poppy seeds within 24 hours prior to check-in for each study period.
   e. Abstain from consumption of alcohol-containing products from 48 hours prior to check-in until after the last sample collection of each study period.
   f. Abstain from foods or beverages containing caffeine, xanthine derivatives or xanthine-related compounds, or energy drinks from 24 hours prior to check-in for each study period.
   g. Abstain from foods or beverages containing grapefruit and/or Seville oranges from 7 days prior to check-in for Period 1 (Stage 1) or Period 4 (Stage 2) and throughout the study.
   h. Abstain from St. Jon's wort from screening and throughout the study.
   i. Abstain from use of tobacco or other nicotine-containing products from screening throughout the study.
   j. Abstain from use of androgens or anabolic steroids from screening throughout the study.

### Check-in (for Each Period)

Subjects will enter the facility for check-in at approximately 4:00 PM on the day before dosing days (check-in on Day −1 for dosing on Day 0, check-in on Day 6 for dosing on Day 7, check-in on Day 13 for dosing on Day 14, check-in on Day 41 for dosing on Day 42, check-in on Day 48 for dosing on Day 49, check-in on Day 55 for dosing on Day 56, and check-in on Day 62 for dosing on Day 63).
1. The inclusion/exclusion criteria will be reviewed and continuing eligibility will be reviewed and assessed.
2. A baseline 12-lead ECG will be performed on Day −1 (Stage 1) and Day 41 (Stage 2).
3. A drug screen and alcohol breathalyzer test will be performed at check-in for each study period. Results must be negative to continue in the study.
4. Any changes in medical history of the subject since the screening visit will be documented and added to the medical history. Clinically significant changes in medical history since Period 1 (Stage 1) or Period 4 (Stage 2) dosing will be recorded as adverse events (AEs).
5. Clinical chemistries and hematology will be collected and assessed for any clinically significant abnormal values.
6. Subjects will be asked about any medications taken since the screening visit or previous study period and adherence to restrictions.
7. A standardized evening meal will be served to the subjects between 5:00 to 7:00 pm or at the normal time of the clinic. An optional snack will be provided at least 10.5 to 11 hours prior to the scheduled dosing (the following day). A supervised fast of at least 10 hours will start after this snack, except for the protocol-defined high-fat meal.
8. The Investigator will review all of the above information and lab results and determine continuing eligibility of the subject prior to each study drug administration.

### Study Period 1 (Stage 1) or Period 4 (Stage 2)

1. Assess the subject's continued qualification for the study based on the inclusion/exclusion criteria and compliance with the requirements for fasting and prescription and nonprescription drugs, herbal medications, and other protocol-outlined food restrictions
2. Prior to the administration of study drug in Period 1 (Stage 1) or Period 4 (Stage 2), subjects will be randomized to a treatment sequence

### In Each Study Period

3. On the morning of Day 0, Day 7, Day 14, Day 42, Day 49, Day 56 and Day 63, subjects will receive Tamsulosin DRS or Flomax (fasting or fed), after a supervised overnight fast of at least 10 hours.
   Food will be controlled and standardized for each housing period and for all subjects. Following an overnight fast of at least 10 hours, randomized subjects will receive a standardized high-fat, high-calorie meal 30 minutes before drug administration. An example meal would be two eggs fried in butter, two strips of bacon, two slices of toast with butter, four ounces of hash brown potatoes and eight ounces of whole milk. Substitutions in this test meal can be made as long as the meal provides a similar amount of calories from protein, carbohydrates, and fat and has comparable meal consistency. Subjects must eat the total contents of this meal in 30 minutes or less.
   At approximately 4.5 and 9.5 hours post dosing of each study period standardized meals will be provided. An evening snack is allowed at approximately 14 hours post dosing of each study period.
4. Prior to the administration of study drug (within 120 minutes prior to dosing), subjects will have vital signs (blood pressure, pulse, temperature, and respiration) performed after sitting for at least 3 minutes prior to the assessment.
5. Blood samples for PK analysis will be taken at the following time points:

| | Analytes |
|---|---|
| Time (hours) | Tamsulosin |
| t₀¹ | X |
| 1 | X |
| 2 | X |
| 3 | X |
| 4 | X |
| 5 | X |
| 6 | X |
| 8 | X |
| 10 | X |
| 12 | X |
| 18 | X |
| 24 | X |
| 30 | X |
| 36 | X |
| 48 | X |

¹The to sample will be drawn within 120 minutes prior to dosing on the dosing day. Based on a reported T½ of 5 hrs.

6. Blood samples for assessment of clinical chemistry and hematology will be collected at screening, at each study check-in and study exit.
   a. Clinical chemistry (blood chemistry) will consist of ALT, albumin, alkaline phosphatase, AST, blood urea nitrogen (BUN), calcium, carbon dioxide (bicarbonate), chloride, creatinine, glucose, inorganic phosphate, magnesium, potassium, sodium, total bilirubin, total protein, uric acid
   b. Hematology will consist of hematocrit (HCT), hemoglobin (HGB), white blood cell (WBC) count, and platelet count and differential
7. Urine samples for urinalysis will be collected at screening and study exit.
   a. Urinalysis will consist of glucose, bilirubin, specific gravity, blood, pH, protein, urobilirubin, nitrite, leucocyte esterase and microscopic assessment (if deemed necessary through evidence of cellular abnormality).
8. All subjects will fast no less than 4 hours after their scheduled dosing time on each dose day. After the 4-hour fast post dose on each dosing day, standard meals will be provided at specified times during the confinement period. Meal plans will be identical for each study period.
9. Monitoring for treatment-emergent adverse events (TEAEs) will begin as soon as the subject receives the first dose of study drug and continue until 48 hours after the last dose or early termination.
10. Vital signs: Seated blood pressure and pulse rate will be measured at approximately 12 hours and 24 hours post-dose [±15 minutes] on Day 0, Day 7, Day 14, Day 42, Day 49, Day 56 and Day 63 (after sitting for at least 3 minutes prior to the assessment), and at any other time deemed medically necessary.
   Release from Clinic or Early Termination

Subjects will be confined in the study center until at least 48 hours after dosing in each period. Subjects will be discharged after all protocol-defined procedures are completed for that period. A reminder about the washout period (food/medication restrictions) and when to return for the next study period (washout of a minimum of 7 days between doses) will be provided.

The following evaluations will be performed after the final blood draw (Day 16 Stage 1 and Day 65 Stage 2) or at early withdrawal:
11. Review of AEs and concomitant medications
12. Physical assessment, including signs of bleeding
13. Fasting laboratory profile (excluding serology and drug/alcohol test)
14. Physical exam, including signs of bleeding
15. 12-lead ECG, supine for at least 3 minutes

The subjects will then be discharged from the study center. If a subject withdraws from the study early, every effort will be made to have the subject remain in the clinic until at least 48 hours after dosing. If they cannot remain in the clinic, every effort will be made to have the subject return to the clinic approximately 48 hours after dosing to have the above evaluations performed.

Total study duration is approximately 17 days for Stage 1 and 25 days for Stage 2 (not including the screening period). Confinement to the clinical site will be from approximately 4:00 PM of the day before dosing in each study period through 48 hours post-dose (a total of approximately 72 hours for each dosing period).

### Criteria for Evaluation:

### Pharmacokinetics

The following noncompartmental PK parameters will be estimated from the plasma concentration-time course data: AUCₜ, AUC_{∞}, Cₘₐₓ, Tₘₐₓ, kₑₗ, Cl/F, t_{1/2}, and Vd/F. No value for AUC_{∞}, km, Cl/F, and t_{1/2}, will be reported for cases that do not exhibit a terminal log-linear phase in the concentration-time data. Single-dose PK parameters will be estimated.

### Safety

Safety assessments will include screening, pre-dose and post-dose vital signs, ECGs, clinical laboratory testing (hematology, blood chemistry, and urinalysis), documentation of AEs, and physical examinations.

### Statistical Methods:

### Pharmacokinetics

Pharmacokinetic parameters (AUCₜ, AUC_{∞}, Cₘₐₓ, Tₘₐₓ, kₑₗ, Cl/F, t_{1/2}, and Vd/F will be estimated following noncompartmental analysis of plasma concentration time-course data for tamsulosin on the days subjects received tamsulosin. Arithmetic means, standard deviations, and coefficients of variation will be calculated for all PK parameters. Additionally, geometric means will be calculated for AUCₜ, AUC_{∞}, and Cₘₐₓ. Analyses of variance (ANOVA) will be performed on the In-transformed PK parameters AUCₜ, AUC_{∞}, and Cₘₐₓ. Each ANOVA will include calculation of least-squares means (LSMs), the differences between adjusted means, and the standard error associated with these differences. Ratios of means will be calculated using the LSM for In-transformed AUCₜ, AUC_{∞}, and Cₘₐₓ.

The 90% confidence interval [CI] for the difference in each analyte will be calculated for the parameters AUCₜ, AUC_{∞}, and Cₘₐₓ using In-transformed concentration data. The CIs will be expressed as a percentage relative to the LSM. The following comparisons will be made:

| | | |
|---|---|---|
| Comparison | Test | Reference |

Stage 1

| | | |
|---|---|---|
| #1 | Tamsulosin DRS (Fed) | FLOMAX ® (Fed) |
| #2 | Tamsulosin DRS (Fasting) | FLOMAX ® (Fed) |
| #3 | Tamsulosin DRS (Fasting) | Tamsulosin DRS (Fed) |

Stage 2

| | | |
|---|---|---|
| #1 | Tamsulosin DRS (Fasting) | FLOMAX ® (Fasting) |
| #2 | Tamsulosin DRS (Fed) | FLOMAX ® (Fed) |
| #3 | Tamsulosin DRS (Fed) | Tamsulosin DRS (Fasting) |

If the 90% Cis for the ratios of geometric least-squares means derived from the analyses of the 1n-transformed PK parameters, AUCₜ, AUC_{∞}, and Cₘₐₓ for comparison #9, outlined above, are within the acceptable range, then no food interaction with the Tamsulosin DRS will be concluded.

### Results

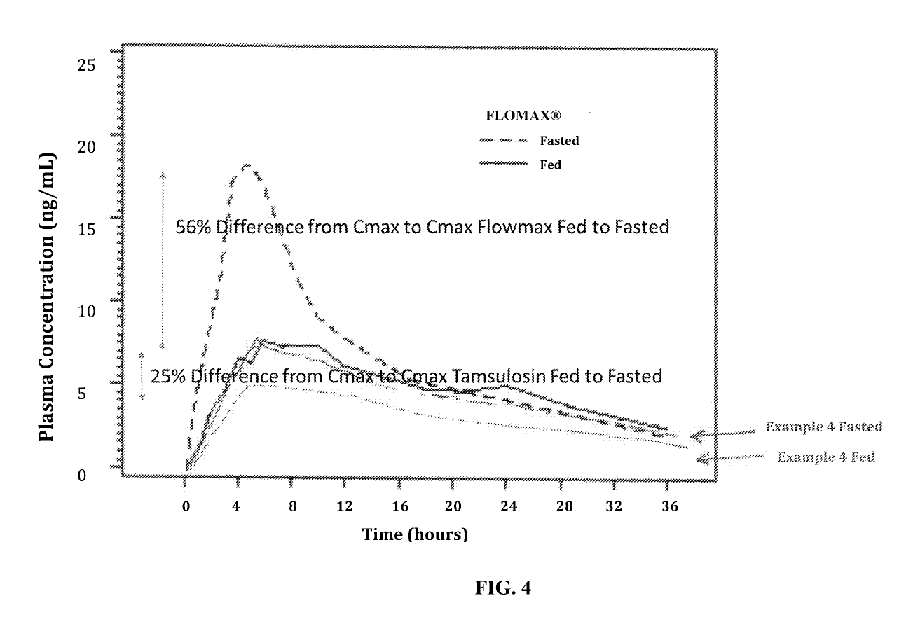

Tamsulosin DRS showed the surprising result that the Tamsulosin DRS under fasted conditions provided a similar PK profile when compared with FLOMAX® fed. The results are shown in FIG. 4. The FLOMAX® package insert dose and administration instructions state “It should be administered approximately one-half hour following the same meal each day”. These instructions are due to the much higher systemic exposure to FLOMAX® when dosed in the fasted state. The study data demonstrated a 56% difference in the Cₘₐₓ for the FLOMAX® fed versus fasted. Tamsulosin DRS in the same study demonstrated only a 25% difference in the Cmax for the fed versus the fasted state. Overall the Tamulosin DRS fasted PK curve was similar to the FLOMAX® fed curve.

Although what has been described hereinabove relates to tamsulosin particularly, persons skilled in the art will appreciate that the methods utilized herein can be used in a similar manner with other active ingredients. The inventive methods applied to such other active ingredients and the resulting dosage forms are considered to be part of the present invention. A list of suitable other actives can be selected from the list Oral Dosage Forms That Should Not Be Crushed 2015 or the list Oral Dosage Forms That Should Not Be Crushed 2016, both of which can be found on the website ismp.org. These lists are incorporated herein by reference. Other active ingredients will be present in the resulting dosage forms in amounts effective for the treatment purposes for which the other active ingredients are utilized. These other active ingredients will be combined with the inventive matrix materials and processed as described herein to yield sachets and the other dosage forms described herein to provide relief against dysphagia and possibly also food effects.

The tamsulosin can also be combined in the embodiments described herein with other active ingredients having a complementing effect against BPH, for example, 5α-reductase inhibitors, especially AVODART® (dutasteride=(1S,3aS,3bS,5aR,9aR,9bS,11aS)-N-[2,5-bis(trifluoromethyl)phenyl]-9a,11a-dimethyl-7-oxo-1,2,3,3a,3b,4,5,5a,6,9b,10,11-dodecahydroindeno[5,4-f]quinoline-1-carboxamide) or a pharmaceutically acceptable salt thereof. Such a combination may have usefulness against androgenetic alopecia. The dosage of dutasteride in such a combination would be 0.5 mg, or another amount effective to treat BPH.

1. A pharmaceutical dosage form comprising an amount effective to treat benign prostatic hyperplasia of a combination of a plurality of particles, wherein each of said particles comprises a mixture of (A) tamsulosin or a physiologically acceptable salt thereof in (B) a controlled release matrix comprising a material selected from the group consisting of cellulose ethers, cellulose esters, and mixtures thereof.

2. The pharmaceutical dosage form according to claim 1, which comprises tamsulosin HCl.

3. The pharmaceutical dosage form according to claim 2, which comprises 0.4 mg of tamsulosin HCl.

4.-5. (canceled)

6. The pharmaceutical dosage form according to claim 1, wherein the controlled release matrix comprises at least one material selected from the group consisting of ethyl cellulose, hydroxypropyl methyl cellulose, hydroxypropyl cellulose, hydroxymethyl cellulose, and derivatives thereof.

7. The pharmaceutical dosage form according to claim 6, wherein the controlled release material comprises at least one material selected from the group consisting of ethyl cellulose, hydroxypropyl methyl cellulose, and derivatives thereof.

8. The pharmaceutical dosage from according to claim 7, wherein the controlled release material comprises a mixture of (i) ethyl cellulose or a derivative thereof, and (ii) hydroxypropyl methyl cellulose or a derivative thereof.

9. The pharmaceutical dosage form according to claim 8, wherein the ethyl cellulose or derivative thereof is selected from the group consisting of ethyl cellulose, and blends of ethyl cellulose and cellulose acetate phthalate; and the hydroxypropyl methyl cellulose or derivative thereof is selected from the group consisting of hydroxypropyl methyl cellulose, hydroxypropyl methyl cellulose acetate succinate, and hydroxypropyl methyl cellulose phthalate.

10. The pharmaceutical dosage form according to claim 1, wherein the at least one mixture of (A) and (B) is homogeneous.

11. The pharmaceutical dosage form according to claim 1, wherein in the pharmaceutical dosage form the amount of (A) is 0.4 mg and the amount of (B) ranges from 100 mg to 400 mg.

12.-17. (canceled)

18. The pharmaceutical dosage form according to claim 1, which is in the form of a sachet.

19. The pharmaceutical dosage form according to claim 18, which further comprises at least one substance selected from the group consisting of sweeteners, surfactants and enteric release materials, optionally in the form of a powder or granules.

20. (canceled)

21. The pharmaceutical dosage form according to claim 1, which is in the form of a capsule.

22. The pharmaceutical dosage form according to claim 21, wherein the capsule has a casing that dissolves in liquid; or has a casing that is breakable or separable.

23. (canceled)

24. The pharmaceutical dosage form according to claim 21, which further comprises at least one substance selected from the group consisting of sweeteners, surfactants and enteric release materials, optionally in the form of a powder or granules.

25. (canceled)

26. The pharmaceutical dosage form according to claim 1, which is in the form of a tablet.

27. The pharmaceutical dosage form according to claim 1, which further comprises a 5α-reductase inhibitor.

28. The pharmaceutical dosage form according to claim 27, wherein the 5α-reductase inhibitor is dutasteride or a pharmaceutically acceptable salt thereof.

29.-30. (canceled)

31. A method of treating benign prostatic hyperplasia in a patient suffering therefrom, said method comprising administering to said patient a pharmaceutical dosage form according to claim 1.

32.-33. (canceled)

34. The pharmaceutical dosage form according to claim 1, which exhibits an in vitro dissolution profile measured at any given time that is at least 80% in agreement with the in vitro dissolution profile of FLOMAX® ((R)-5-(2-{[2-(2-Ethoxyphenoxy)ethyl]amino}propyl)-2-methoxybenzene-1-sulfonamide hydrochloride) shown in FIG. 3 at the same given time.

35. The pharmaceutical dosage form according to claim 1, which exhibits an in vivo plasma concentration that is at any given time at least 80% in agreement with the in vivo plasma concentration of FLOMAX® ((R)-5-(2-{[2-(2-Ethoxyphenoxy)ethyl]amino}propyl)-2-methoxybenzene-1-sulfonamide hydrochloride) “Fed” shown in FIG. 4 at the same given time.
